(19) United States
(12) Patent Application Publication
(10) Pub. No.: US 2015/0203907 A1
(43) Pub. Date: Jul. 23, 2015

(54) GENOME CAPTURE AND SEQUENCING TO DETERMINE GENOME-WIDE COPY NUMBER VARIATION

(71) Applicant: FLORIDA STATE UNIVERSITY RESEARCH FOUNDATION, TALLAHASSEE, FL (US)

(72) Inventors: DAVID M. GILBERT, TALLAHASSEE, FL (US); JONATHAN H. DENNIS, TALLAHASSEE, FL (US); TAKAYO SASAKI, TALLAHASSEE, FL (US)

(21) Appl. No.: 14/598,261

(22) Filed: Jan. 16, 2015

Related U.S. Application Data
(60) Provisional application No. 61/928,473, filed on Jan. 17, 2014.

(51) Int. Cl. C12Q 1/68

(52) U.S. Cl. CPC C12Q 1/6869

(57) ABSTRACT

Provided herein is a capture library for target enrichment of sequences of interest from a genome DNA sample. The capture library comprises a plurality of capture oligos tiling a plurality of capture regions evenly-spaced along a genome. Each two adjacent capture regions of the plurality capture regions are separated by a spacing of about 6 to about 14 kilobases in length. The plurality of capture regions has a size of about 150 base pairs in length. Further, each capture oligo of the plurality of capture oligos comprises average 70 nucleotides in length. The capture libraries are suitable for enriching about 150 base pairs region approximately every 10 kilobases in a genome DNA. This capture library can be used to measure replication timing and copy number variation in human pediatric acute lymphocytic leukemia samples, and is also broadly applicable to any CNV application.

### CROSS-REFERENCE TO RELATED APPLICATIONS

This application claims benefit of priority to U.S. Provisional Patent Application No. 61/928,473 to Gilbert et al., entitled “Genome Capture and Sequencing to Determine Genome-Wide Copy Number Variation,” filed Jan. 17, 2014. The entire contents and disclosure of this patent application are incorporated herein by reference in its entirety.

This application makes reference to the following U.S. patents and U.S. patent applications: U.S. Provisional Patent Application No. 60/969,399, entitled “Method for Identifying Cells Based on DNA Replication Domain Timing,” filed Aug. 31, 2007; U.S. patent application Ser. No. 12/200,186, entitled “Method for Identifying Cells Based on DNA Replication Domain Timing Profiles,” filed Aug. 28, 2008; U.S. now U.S. Pat. No. 8,728,979, issued May 20, 2014; Provisional Patent Application No. 61/489,467, entitled “Genome-Scale Analysis of Replication Timing: From Bench to Bioinformatics,” filed May 24, 2011; U.S. Provisional Patent Application No. 61/527,771, entitled “Fingerprint for Cell Identity and Pluripotency,” filed Aug. 26, 2011; U.S. Provisional Patent Application No. 61/580,401, entitled “Replication Timing Profiles for Leukemia and Other Cancers,” filed Dec. 27, 2011; U.S. Continuation-in-Part patent application Ser. No. 13/479,686, entitled “Genome-Scale Analysis of Replication Timing,” filed May 24, 2012; U.S. patent application Ser. No. 13/595,017, entitled “Fingerprint for Cell Identity and Pluripotency, filed Aug. 27, 2012; U.S. patent application Ser. No. 13/726,803, entitled “Replication Timing Profiles for Leukemia and Other Cancers,” filed Dec. 26, 2012, now U.S. Pat. No. 8,725,423, issued May 13, 2014; U.S. Divisional patent application Ser. No. 14/267,953, entitled “Method for Identifying Cells Based on DNA Replication Domain Timing,” filed May 2, 2014; and U.S. Provisional Patent Application No. 61/928,473 to Gilbert et al., entitled “Genome Capture and Sequencing to Determine Genome-Wide Copy Number Variation,” filed Jan. 17, 2014. The entire contents and disclosure of these patent applications are incorporated herein by reference in their entirety.

### BACKGROUND

1. Field of the Invention

The present invention relates to the analysis of nucleic acids and to targeted sequencing

2. Related Art

Genomic abnormalities are often associated with various genetic disorders degenerative diseases, and cancer. For example, the deletion or multiplication of copies of genes and the deletion or amplifications of genomic fragments or specific regions are common occurrences in cancer. The identification and cloning of specific genomic regions associated with cancer and various genetic disorder is therefore of interest both to the study of tumorigenesis and in developing better means of diagnosis and prognosis. Next-generation sequencing enables researchers to obtain large amounts of data more rapidly and efficiently. However, the sequence output generated by traditional next-generation sequencing far exceeds the analysis requirements for a single sample. It is still too expensive to deeply sequence many samples to find most genetic variants. Targeting sequencing specific genomic regions of interest is a key to advancing our knowledge of genomic variation and its relationship to disease.

### SUMMARY

According to a first broad aspect, the present invention provides a product comprising a capture library comprising a plurality of capture oligos, wherein the plurality of capture oligos tiles a plurality of capture regions approximately evenly-spaced along a genome, wherein the plurality of capture regions comprises about 68 to about 196 base pairs (bp) with an average of about 150 bp, wherein each two adjacent capture regions of the plurality capture regions are separated by a spacing of about 6 to about 14 kilobases (kb), and wherein the product is a comparative genomic hybridization capture library.

### BRIEF DESCRIPTION OF THE DRAWINGS

The accompanying drawings, which are incorporated herein and constitute part of this specification, illustrate exemplary embodiments of the invention, and, together with the general description given above and the detailed description given below, serve to explain the features of the invention.

FIG. 1 is a schematic illustration of capture regions evenly-spaced along a genome according to one embodiment of the present invention.

FIG. 2 is a schematic illustration of design of capture probes according to one embodiment of the present invention.

FIG. 3 is a schematic illustration of design of capture probes according to one embodiment of the present invention.

FIG. 4 is a schematic illustration of general steps for performing the target enrichment with the use of the capture probes designed herein according to one embodiment of the present invention.

FIG. 5 is a schematic illustration of a strategy for target enrichment according to one embodiment of the present invention.

FIG. 6 is a graph of relative quantity for target and non-target loci of pre-/post-capture samples (showing post-capture enrichment evaluation by qPCR) according to one embodiment of the present invention.

FIG. 7 shows screenshots from UCSC Genome browser with custom tracks (capture regions and actual reads) (showing distribution of sequence reads with and without capture; reads from non-captured sample distribute randomly while reads from captured sample distribute in 150 bp capture region shown in FIG. 1 according to one embodiment of the present invention.

FIG. 8 shows an enlarged representative region from FIG. 7 (distribution of sequence reads) according to one embodiment of the present invention.

FIG. 9 shows a comparison of raw data (before scaling) dynamic range from E/L, non-capture S/G1, captured S/G1 repli-seq according to one embodiment of the present invention.

FIG. 10 is an illustration of a product comprising a capture library contained in a container according to one embodiment of the present invention.

### DETAILED DESCRIPTION OF THE PREFERRED EMBODIMENTS

### Definitions

Where the definition of terms departs from the commonly used meaning of the term, applicant intends to utilize the definitions provided below, unless specifically indicated.

Generally, nomenclatures utilized in connection with, and techniques of, cell and tissue culture, molecular biology, and protein and oligo- or polynucleotide chemistry and hybridization described herein are well known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Green and Sambrook, Molecular Cloning: A Laboratory Manual (Fourth ed., Cold Spring Harbor Laboratory Press, Cold Spring Harbor, N.Y. 2012). The nomenclatures utilized in connection with, and the laboratory procedures and techniques described herein are well known and commonly used in the art.

For purposes of the present invention, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

For purposes of the present invention, a value or property is “based on” or “derived from” a particular value, property, the satisfaction of a condition or other factor if that value is derived by performing a mathematical calculation or logical decision using that value, property, condition or other factor.

For purposes of the present invention, it should be noted that the singular forms, “a,” “an” and “the,” include reference to the plural unless the context as herein presented clearly indicates otherwise. For example, as used herein, “a” or “an” also may refer to “at least one” or “one or more.” Also, in this application, the use of the singular includes the plural unless specifically stated otherwise. For example, the use of “comprise”, “comprises”, “comprising”, “contain”, “contains”, “containing”, “include”, “includes”, and “including” are not intended to be limiting.

For purposes of the present invention, the term “adjacent” refers to two regions or segments of interest on each side of a DNA sequence gap. For example, two adjacent regions of interest, for example, adjacent capture regions, on a genome are two segments of DNA of interest separated by a gap and there is no other region of interest exists between these two capture regions.

For purposes of the present invention, the term “alteration” refers to a change, modification or adjustment or deviation from a standard.

For purposes of the present invention, the term “array” and the term “microarray” refer interchangeably to a field or array of a multitude of spots corresponding to nucleic acid probes or oligonucleotides for all or at least a portion of the genome of a species placed on a support or substrate to allow for simultaneous detection and/or quantification of nucleic acid molecules present in one or more sample(s) by hybridization as commonly understood in the art.

For purposes of the present invention, the term “beads” refers to streptavidin-coated particles that are used to recover biotinylated oligos from the solution. For example, the beads may be streptavidin-coated magnetic particles.

For purposes of the present invention, the term “biomolecule” refers to any molecule that is produced by a biological organism, including large polymeric molecules such as proteins, polysaccharides, lipids, and nucleic acids (DNA and RNA) as well as small molecules such as primary metabolites, secondary metabolites, and other natural products.

For purposes of the present invention, the term “capture library” or the term “sequencing library” refers to a collection or a pool of capture oligonucleotides or capture probes. In a capture library or sequencing library, each capture oligonucleotide may be labeled with a bead, such as streptavidin-coated magnetic beads.

For purposes of the present invention, the term “capture material” refers to any suitable type of materials that provide a known binding capacity, resulting in a substantially constant amount of captured DNA per fixed amount of capture material. For example, the capture material may be streptavidin, streptavidin-coated beads, or streptavidin-coated magnetic beads. In an embodiment where streptavidin is used as a capture material, the nucleic acids in testing genomic DNA library or reference genomic DNA library may be biotinylated to facilitate binding of the nucleic acids to the capture material.

For purpose of the present invention, the term “capture oligonucleotide,” the term “capture probe,” and the term “capture oligos” refer to single-stranded oligonucleotide used for capturing an interested candidate genomic region during target enrichment. Capture oligos can be synthesized chemically at high quality by column-based synthesis, or at relatively lower quality and cost using programmable microarrays. Capture oligos or capture probes may be further labeled or bound to certain capture substrates such as beads for pulling captured sequences from uncaptured sequences.

For purposes of the present invention, the term “capture region” refers to a selected sequence or region from full complexity genome.

For purposes of the present invention, the term “capture spacing” refers to the distance between two adjacent capture regions

For purposes of the present invention, the term “cell type” refers to the kind, identity and/or classification of cells according to any and all criteria, such as their tissue and species of origin, their differentiation state, whether or not (and in what manner) they are normal or diseased, etc. For example, the term “cell type” may refer separately and specifically to any specific kind of cell found in nature, such as an embryonic stem cell, a neural precursor cell, a myoblast, a mesodermal cell, etc. Such a list of possible cell types is meant herein to be unlimited.

For purposes of the present invention, the term “comparative genomic hybridization (CGH)” refers to a screening technique that can identify regions of gain and loss within the whole genome in a single experiment. CGH is a method for analyzing copy number variations (CNVs) relative to ploidy level in the DNA of a test sample compared to a reference sample, without the need for culturing cells. The aim of this technique is to quickly and efficiently compare two genomic DNA samples arising from two sources, which are most often closely related, because it is suspected that they contain differences in terms of either gains or losses of either whole chromosomes or subchromosomal regions (a portion of a whole chromosome). This technique was originally developed for the evaluation of the differences between the chromosomal complements of solid tumor and normal tissue, and has an improved resolution of 5-10 megabases compared to the more traditional cytogenetic analysis techniques of giemsa banding and fluorescence in situ hybridization (FISH) which are limited by the resolution of the microscope utilized. CGH is often used in diagnostics to compare differences between types of DNA, such as normal cells vs. cancer cells.

For purposes of the present invention, the term “complementary” or the term “complementarity” refers to polynucleotides (e.g., a sequence of nucleotides) related by the base-pairing rules. For example, for the sequence 5′-A-G-T-3′ is complementary to the sequence 3′-T-C-A-5′. Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods which depend upon binding between nucleic acids.

For purposes of the present invention, the term “comprising”, the term “having”, the term “including,” and variations of these words are intended to be open-ended and mean that there may be additional elements other than the listed elements.

For purposes of the present invention, the term “copy-number variations (CNVs)” refers to a form of structural variation of the DNA of a genome that results in the cell having an abnormal or, for certain genes, a normal variation in the number of copies of one or more sections of the DNA. CNVs correspond to relatively large regions of the genome that have been deleted (fewer than the normal number) or duplicated (more than the normal number) on certain chromosomes. For example, the chromosome that normally has sections in order as A-B-C-D might instead have sections A-B-C-C-D (a duplication of “C”) or A-B-D (a deletion of “C”). This variation accounts for roughly 12% of human genomic DNA and each variation may range from about one kilobases (1,000 nucleotide bases) to several megabases in size. CNVs contrast with single-nucleotide polymorphisms (SNPs), which affect only one single nucleotide base.

For purposes of the present invention, the term “coverage,” the term “read depth,” and the term “depth” refer to the average number of reads representing a given nucleotide in the reconstructed sequence. It can be calculated from the length of the original genome (G), the number of reads (N), and the average read length (L) as N×L/G. For example, a hypothetical genome with 2,000 base pairs reconstructed from 8 reads with an average length of 500 nucleotides will have about two times (2×) of redundancy. This parameter also enables one to estimate other quantities, such as the percentage of the genome covered by reads (sometimes also called coverage). Usually, a high coverage in shotgun sequencing is desired because it can overcome errors in base calling and assembly.

For purposes of the present invention, the term “DNA fragmentation” refers to the separation or breaking of DNA strands into pieces. DNA fragmentation is often necessary prior to library construction or subcloning for DNA sequences. A variety of methods involving the mechanical breakage of DNA have been employed where DNA is fragmented by intentional laboratory personnel. Such methods include sonication, needle shear, nebulisation, point-sink shearing and passage through a pressure cell. For example, sonication is a type of hydrodynamic shearing, which subjects DNA to Hydrodynamic shearing by exposure to brief periods of sonication, usually resulting in 700 bp fragments. According to the embodiments of the present invention, DNA fragmentation may be achieved by any type of shearing method.

For purposes of the present invention, the term “enrichment specificity” and the term “sequence capture specificity” refer to that the fraction of nucleic acid fragments obtained at the end of an experiment that are explicitly targeted for capture at the beginning This is typically measured as the percentage of sequence reads, or sequenced bases, from an experiment that align with the targeted portion of a reference sequence. Along with sequence coverage uniformity across the target, enrichment specificity is a major determinant of overall process efficiency. For example, an experiment yielding 35% of sequence reads mapping to the intended target (i.e. a 35% on-target rate) would require approximately twice much sequencing to get the same amount of useful data as an experiment with a 70% on-target rate for that same target.

For purposes of the present invention, the term “dNTP” refers to deoxynucleotidetriphosphate, where the nucleotide is any nucleotide, such as A, T, C, G or U.

For purposes of the present invention, the term “epigenetic signature” and the term “epigenetic signatures” broadly refer to any manifestation or phenotype of cells of a particular cell type that is believed to derive from the chromatin structure of such cells.

For purposes of the present invention, the term “epigenetics,” the term “epigenetic markers” and the term “epigenetic parameters” refer to chemical modifications of DNA, histones or other chromatin-associated molecules that impart changes in gene expression, such as methylation, acetylation, ubiquitylation, etc. However, the terms “epigenetics,” “epigenetic markers” and “epigenetic parameters” may refer to any changes in chromatin structure that affect gene expression apart from DNA sequence. For example, the terms “epigenetics,” “epigenetic markers” and “epigenetic parameters” may refer to incorporation of histone variants or chromosomal remodeling by enzymes.

For purposes of the present invention, the term “enrichment factor” refers to a ration of copy number of captured region in a post-capture library to copy number of captured region in a pre-capture library.

For purposes of the present invention, the term “evenly spaced” refers to a series of capture regions in which each two adjacent capture regions are separated by a spacing between a range of length but have a certain average distance. For example, the spacing between two adjacent capture regions may be about 6 to about 14 kilobases (kb) but have an average distance of 10 kb.

For purposes of the present invention, the term “FASTQ” refers to a text-based format for storing both a biological sequence (usually nucleotide sequence) and its corresponding quality scores. Both the sequence letter and quality score are encoded with a single ASCII character for brevity. It was originally developed at the Wellcome Trust Sanger Institute to bundle a FASTA sequence and its quality data, but has recently become the de facto standard for storing the output of high throughput sequencing instruments such as the Illumina Genome Analyzer.

For purposes of the present invention, the term “fluorescence in situ hybridization (FISH)” refers to a cytogenetic technique developed by biomedical researchers in the early 1980s that is used to detect and localize the presence or absence of specific DNA sequences on chromosomes. FISH uses fluorescent probes that bind to only those parts of the chromosome with which they show a high degree of sequence complementarity. Fluorescence microscopy can be used to find out where the fluorescent probe is bound to the chromosomes. FISH is often used for finding specific features in DNA for use in genetic counseling, medicine, and species identification. FISH can also be used to detect and localize specific RNA targets (mRNA, lncRNA and miRNA) in cells, circulating tumor cells, and tissue samples. In this context, it can help define the spatial-temporal patterns of gene expression within cells and tissues.

For purposes of the present invention, the term “fragment DNA” refers to separating or breaking DNA strands into pieces. It can be intentional by laboratory personnel or the cells, or it can be spontaneous.

For purposes of the present invention, the term “genome” refers to genetic material of an organism. It is encoded either in DNA or, for many types of viruses, in RNA. The genome includes both the genes and the non-coding sequences of the DNA/RNA.

For purposes of the present invention, the term “genome region” refers to a segment of nucleic acids or a fragment of nucleic acids on a genome.

For purposes of the present invention, the term “genome-wide” and the term “whole genome” may refer interchangeably to the entire genome of a cell or population of cells. Alternatively, the terms “genome-wide” or “whole genome” may refer to most or nearly all of the genome. For example, the terms “genome-wide” or “whole genome” may exclude a few portions of the genome that are difficult to sequence, do not differ among cells or cell types, are not represented on a whole genome array, or raise some other issue or difficulty that prompts exclusion of such portions of the genome.

For purposes of the present invention, the term “genomic array” is an array having probes and/or oligonucleotides corresponding to both coding and non-coding intergenic sequences for at least a portion of a genome and may include the whole genome of an organism. For example, a “genomic array” may have probes and/or oligonucleotides for only those portions of a genome of an organism that correspond to replication timing fingerprint(s) or informative segments of fingerprint(s). The term “genomic array” may also refer to a set of nucleic acid probes or oligonucleotides representing sequences that are about evenly spaced along the length of each chromosome or chromosomal segment. However, even spacing of probes may be dispensable with very high-density genomic arrays (i.e., genomic arrays having an average probe spacing of much less than about 6 kb).

For purposes of the present invention, the term “hybridization” and the term “hybridizes” refers to a process in which a nucleic acid strand anneals to and forms a stable duplex, either a homoduplex or a heteroduplex, under normal hybridization conditions with a second complementary nucleic acid strand, and does not form a stable duplex with unrelated nucleic acid molecules under the same normal hybridization conditions. The formation of a duplex is accomplished by annealing two complementary nucleic acid strands in a hybridization reaction.

For purposes of the present invention, the term “high resolution array” and the term “high resolution genomic array” refer to a genomic array having sufficient resolution to provide enough information to generate a smooth replication timing profile to reliably determine the exact positions, lengths, boundaries, etc., of the replication timing domains. The term “high resolution array” or “high resolution genomic array” may correspond to the whole genome or a substantial portion of a genome of a particular cell or population of cells. The term “high resolution array” or “high resolution genomic array” may also refer to a genomic array having an average probe spacing of about 6 kilobases (kb) or less.

For purposes of the present invention, the term “kit” refers to a package of product. For example, a kit of capture library is a package including a plurality of capture oligos with or without other capture materials or substrates. In some examples, a kit of capture library may encompass a plurality of capture oligos and a plurality of capture substrates such as capture beads, wherein the plurality of beads may be directly coupled to the plurality of capture oligos. In some examples, capture oligos and beads may be separately packed in a kit so that capture beads may be applied to the plurality of capture oligos at a time according to needs.

For purposes of the present invention, the term “library” or “nucleic acid library” refers to a collection of nucleic acid molecules or DNA fragments. For example, “genomic library” is a generic term referring to any collection of sequences representing an entire genome; “sequencing library” is a library that is specifically constructed to be amenable to running through a sequencing machine; and “capture library” is a collection of oligonucleotides that constitute a part of the genome that are going to be captured.

For purposes of the present invention, the term “machine-readable medium” refers to any mechanism that stores information in a form accessible by a machine such as a computer, network device, personal digital assistant, manufacturing tool, any device with a set of one or more processors, etc. For example, a machine-readable medium may be a recordable/non-recordable medium (e.g., a read-only memory (ROM), a random access memory (RAM), a magnetic disk storage medium, an optical storage medium, a flash memory device, etc.), a bar code, an RFID tag, etc.

For purposes of the present invention, the term “mammalian cells” refers to a population of cells that are, or were, originally derived from a mammalian organism. The term “mammalian cells” may include primary cells derived from a mammalian species or a cell line originally derived from a mammalian species. The term “mammalian cells” may refer to a homogeneous population of cells from a mammalian organism.

For purposes of the present invention, the term “multiplex amplification” refers to selective and non-random amplification of two or more target sequences within a sample using at least one target-specific primer. In some embodiments, multiplex amplification is performed such that some or all of the target sequences are amplified within a single reaction vessel. The “plexy” or “plex” of a given multiplex amplification refers to the number of different target-specific sequences that are amplified during that single multiplex amplification. In some embodiments, the plexy can be about 12-plex, 24-plex, 48-plex, 96-plex, 192-plex, 384-plex, 768-plex, 1536-plex, 3072-plex, 6144-plex or higher.

For purposes of the present invention, the term “next generation sequencing (NGS)” refers to sequencing technologies having high-throughput sequencing as compared to traditional Sanger- and capillary electrophoresis-based approaches, wherein the sequencing process is performed in parallel, for example producing thousands or millions of relatively small sequence reads at a time. Some examples of next generation sequencing techniques include, but are not limited to, sequencing by synthesis, sequencing by ligation, and sequencing by hybridization. These technologies produce shorter reads (anywhere from 25-500 bp) but many hundreds of thousands or millions of reads in a relatively short time.

For purposes of the present invention, the term “non-repetitive regions in genome” refers to regions that contain no repeated sequences. Repeated sequences are patterns of nucleic acids (DNA or RNA) that occur in multiple copies throughout the genome.

For purposes of the present invention, the term “nucleic acid” refers to any nucleic acid molecule, including, without limitation, DNA, RNA and hybrids thereof. The nucleic acid bases that form nucleic acid molecules can be the bases A, C, G, T and U, as well as derivatives thereof. Derivatives of these bases are well known in the art. The term should be understood to include, as equivalents, analogs of either DNA or RNA made from nucleotide analogs. The term as used herein also encompasses cDNA, that is complementary, or copy, DNA produced from an RNA template, for example by the action of reverse transcriptase.

For purposes of the present invention, the term “nucleic acid sequencing data,” the term “nucleic acid sequencing information,” the term “nucleic acid sequence,” the term “genomic sequence,” the term “genetic sequence,” the term “fragment sequence,” or the term “nucleic acid sequencing read” refers to any information or data that is indicative of the order of the nucleotide bases (e.g., adenine, guanine, cytosine, and thymine/uracil) in a molecule (e.g., whole genome, whole transcriptome, exome, oligonucleotide, polynucleotide, fragment, etc.) of DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, etc.

For purposes of the present invention, the term “off-target reads” refers to sequence reads mapped outside of the capture region.

For purposes of the present invention, the term “oligonucleotide” and the term “oligo” refers to short, single-stranded multimer of nucleotides.

For purposes of the present invention, the term “phenotype” refers to the detectable characteristics of a cell or organism which are a manifestation of gene expression.

For purposes of the present invention, the term “plurality” refers to contain at least 2 members.

For purposes of the present invention, the term “polynucleotide”, the term “nucleic acid”, or the term “oligonucleotide” refers to a molecule comprised of two or more deoxyribonucleotides or ribonucleotides. The term ‘nucleic acid’ includes DNA (such as cDNA or genomic DNA) and RNA (such as mRNA or microRNA) that is single- or double-stranded, optionally containing synthetic, non-natural or altered nucleotide bases, synthetic nucleic acids and combinations thereof.

For purposes of the present invention, the term “population of cells” refers to a homogeneous group or population of cells. The term “population of cells” may also include a single cell in culture having the potential to grow and divide into a plurality of homogeneous cells under appropriate culturing conditions.

For purposes of the present invention, the term “primary cell” refers to a cell or cells isolated from a tissue of an organism and placed in culture. The “primary cell” may be derived from any tissue of any organism, such as a mammalian organism. The term “primary cell” includes any cell or cells that may be isolated from a tissue of an organism to create a reasonably homogeneous population of cells, such as by first creating single-cell suspensions.

For purposes of the present invention, the term “purified” refers to molecules, either nucleic or amino acid sequence, that are removed from their natural environment, isolated or separated. An “isolated nucleic acid sequence” may therefore be a purified nucleic acid sequence. “Substantially purified” molecules are at least 60% free, preferably at least 75% free, and more preferably at least 90% free from other components with which they are naturally associated. As used herein, the term “purified” or “to purify” also refer to the removal of contaminants from a sample. The removal of contaminating proteins results in an increase in the percent of polypeptide of interest in the sample.

For purposes of the present invention, the term “replication timing” refers to the order in which DNA is duplicated during the synthesis phase of the cell cycle and is correlated with the expression of genes and the structure of chromosomes.

For purposes of the present invention, the term “replication timing domain” refers to a contiguous region of a chromosome of a cell or population of cells having roughly the same (i.e., early vs. late) replication timing, such as a contiguous region of a chromosome of a cell or population of cells having roughly the same replication timing ratio value.

For purposes of the present invention, the term “replication timing profile” refers to a series of values for replication timing (e.g., early versus late S-phase replication timing) along the length of at least a segment of one or more chromosome(s) within a genome. For example, the “replication timing profile” may be expressed as a series of replication timing ratio values, such as early/late S-phase replication or late/early S-phase replication, along the length of at least a segment of one or more chromosome(s), which may further be expressed on a logarithmic scale. Alternatively, the “replication timing profile” may refer to a ratio of the amounts of S-phase DNA to G1-phase DNA from a population of asynchronously dividing cells along the length of at least a segment of one or more chromosome(s), which further may be expressed on a logarithmic scale, with a higher ratio indicating earlier replication and a lower ratio indicating later replication. The term “replication timing profile” may include a replication timing fingerprint for a particular cell type or a set of replication timing profiles for informative segments of a replication timing fingerprint for a particular cell type. The term “replication timing profile” further may include a replication timing profile differential between any combinations of: (1) one or more replication timing profile(s); (2) a replication timing fingerprint; and/or (3) one or more informative segment(s) of a replication timing fingerprint(s). The “replication timing profile” may be determined, for example, by quantifying an amount of replicated DNA in a sample from a population of cells by measuring fluorescently labeled DNA, by sequencing, etc.

For purposes of the present invention, the term “replication timing ratio” refers to a ratio value for the timing of replication at a particular locus of a chromosome within the genome of a cell. For example, the “replication timing ratio” may be a ratio of the extent of replication in early S-phase cells divided by the extent of replication in late S-phase cells, or vice versa, at a given locus. Alternatively, the replication timing ratio may be expressed on a logarithmic scale, such as log2(early/late) or log2(late/early). Alternatively, for example, the term “replication timing ratio” may refer to the ratio of the extent of replicated DNA in S-phase cells to the amount of DNA in G1-phase cells. The extent of replication or the amount of DNA may be measured, for example, by the fluorescence intensity of an attached label.

For purposes of the present invention, the term “resolution” with respect to arrays refers to average or median capture spacing, for example, a given size of DNA region that would reside between adjacent array elements along the length of one or more chromosomes. In some situations, resolution may refer to the sensitivity to determine segmental copy number alterations. In general, the more probes and/or oligonucleotides along a given length of a chromosome, the greater or higher the resolution may be for such length of a chromosome, assuming roughly equal spacing. Therefore, the terms “density” and “probe density” for an array are directly related to the term “resolution,” since a greater or higher probe density along a given length of a chromosome would generally result in greater or higher resolution for the same length of a chromosome. Conversely, the term “spacing” or “probe spacing” is inversely related to gene density and resolution for an array, since a lower or reduced spacing on average between probes and/or oligonucleotides on the array as a function of chromosomal position would generally result in greater or higher resolution or probe density. For example, an array having an average “probe spacing” of about 6 kb or less along a length of a chromosome would have a “probe density” or “resolution” of about 6 kb or higher for such length of chromosome.

For purposes of the present invention, the term “sequence capture” or the term “genome capture” refers to a procedure to enrich selected sequences/genomic regions from full complexity genomic DNA in a single step. By performing sequence capture to the sequence library (pool) before sequencing, only genomic regions/genes of interest will be sequenced from originally whole genome, transcriptome, etc. libraries. Commercially designed capture libraries such as exome, exome +UTR, etc. are available. Custom-order capture libraries are available as well.

For purposes of the present invention, the term “sequencing library” refers to a collection of nucleic acid fragments from a genome, sheard to even length and added adpator and index sequence on both ends for NGS.

For purposes of the present invention, the term “sequencing run” refers to any step or portion of a sequencing experiment performed to determine some information relating to at least one biomolecule (e.g., nucleic acid molecule).

For purposes of the present invention, the term “Single Nucleotide Polymorphism (SNP)” refers to a DNA sequence variation occurring commonly within a population (e.g. 1%) in which a Single Nucleotide—A, T, C or G—in the genome (or other shared sequence) differs between members of a biological species or paired chromosomes. SNP density can be predicted by the presence of microsatellites: AT microsatellites in particular are potent predictors of SNP density, with long (AT)(n) repeat tracts tending to be found in regions of significantly reduced SNP density and low GC content.

For purposes of the present invention, the term “size” as used for a nucleic acid sequence refers to the length of the nucleic acid sequence. A “window size” refers to the length of a DNA fragment or region. For example, “a size of DNA window is 10 kb” indicates that a region of DNA sequence has a length of 10 kb nucleic acids.

For purposes of the present invention, the term “solution” refers to a homogeneous mixture composed of only one phase. When the term “solution” is used in association with a reaction, such as “a reaction in solution,” it means that the solution consists of two or more substances dissolved in a liquid form and the term is used in contrast to “reaction on a solid phase.” For example, “solution-based capture” is a target enrichment strategy to capture genomic regions of interest in which a pool of oligonucleotides, i.e., capture probes or capture oligos, is synthesized and dissolved with a fragmented genomic DNA sample in a solution, wherein the capture oligos hybridize to the fragmented genomic DNA sample.

For purposes of the present invention, the term “spot” refers to an area, region, etc. of the surface of a support, substrate, etc., having identical, similar, and/or related nucleic acid probe or oligonucleotide sequences. Such nucleic acid probes may include vectors, such as BACs, PACs, etc. Each “spot” may be arranged so that it does not touch, become indistinguishable from or become continuous with other adjacent spots.

For purposes of the present invention, the term “storage” and the term “storage medium” refer to any form of storage that may be used to store bits of information. Examples of storage include both volatile and non-volatile memories such as ERAM, flash memory, floppy disks, Zip™ disks, CD-ROM, CD-R, CD-RW, DVD, DVD-R, DVD+R, hard disks, optical disks, etc.

For purposes of the present invention, the term “system” refers to a set of components, real or abstract, comprising a whole where each component interacts with or is related to at least one other component within the whole.

For purposes of the present invention, the term “target-enrichment” refers to selectively capture genomic regions of interest from a DNA sample prior to sequencing. Several target-enrichment strategies have been developed since the original description of the direct genomic selection (DGS) method in 2005. For example, Polymerase chain reaction (PCR) is one of the most widely used enrichment strategies for over 20 years. Other enrichment strategies, such as molecular inversion probes (MIP), hybrid capture, in-solution capture, etc., have also development recently.

For purposes of the present invention, the term “target region” refers to an interested part in genomic DNA.

For purposes of the present invention, the term “targeted sequencing” refers to efficient sequencing a small subset of the genome. In clinical settings, sequencing a subset of the genome not only reduce costs, but also focuses on the relevant regions. The main challenge for clinical targeted resequencing methods is obtaining complete and uniform coverage of all target regions. Some popular methods for target enrichment rely on lengthy and inefficient hybrid capture or multiplexed PCR techniques, resulting in lower coverage and more off-target sequencing reads.

For purposes of the present invention, the term “type of leukemia” refers to any distinguishable type of leukemia. The types of leukemia can be grouped based on how quickly the disease develops and gets worse. For example, leukemia may be considered either chronic (which usually gets worse slowly) or acute (which usually gets worse quickly). In the case of chronic leukemia, early in the disease, the leukemia cells can still do some of the work of normal white blood cells. People may not have any symptoms at first. Doctors often find chronic leukemia during a routine checkup—before there are any symptoms. Slowly, chronic leukemia gets worse. As the number of leukemia cells in the blood increases, people get symptoms, such as swollen lymph nodes or infections. When symptoms do appear, they are usually mild at first and get worse gradually. In the case of acute leukemia, the leukemia cells can't do any of the work of normal white blood cells. The number of leukemia cells increases rapidly. Acute leukemia usually worsens quickly. The types of leukemia also can be grouped based on the type of white blood cell that is affected. Leukemia can start in lymphoid cells or myeloid cells. Leukemia that affects lymphoid cells is called lymphoid, lymphocytic, or lymphoblastic leukemia. Leukemia that affects myeloid cells is called myeloid, myelogenous, or myeloblastic leukemia. There are four common types of leukemia: (1) chronic lymphocytic leukemia (CLL), (2) chronic myeloid leukemia (CML), (3) acute lymphocytic (lymphoblastic) leukemia (ALL) and (4) acute myeloid leukemia (AML). CLL affects lymphoid cells and usually grows slowly. It accounts for more than 15,000 new cases of leukemia each year. Most often, people diagnosed with the disease are over age 55. It almost never affects children. CML affects myeloid cells and usually grows slowly at first. It accounts for nearly 5,000 new cases of leukemia each year. It mainly affects adults. ALL affects lymphoid cells and grows quickly. It accounts for more than 5,000 new cases of leukemia each year. ALL is the most common type of leukemia in young children. It also affects adults. AML affects myeloid cells and grows quickly. It accounts for more than 13,000 new cases of leukemia each year. It occurs in both adults and children.

For purposes of the present invention, the term “whole genome sequencing,” the term “full genome sequencing”, the term “complete genome sequencing”, and the term “entire genome sequencing” refer to a laboratory process that determines the complete DNA sequence of an organism's genome at a single time. This entails sequencing all of an organism's chromosomal DNA as well as DNA contained in the mitochondria and, for plants, in the chloroplast.

For purposes of the present invention, the term “window” refers to a range or a frame of nucleic acid sequence along a genome region under study.

It is to be understood that both the foregoing general description and the following detailed description are exemplary and explanatory only and are not restrictive of the present teachings.

### Description

Embodiments in the present invention provide a customer designed library comprising a plurality of capture probes that are useful in target enrichment of whole genomic DNA fractions in solution. Solution-based sequence capture with the use of the capture libraries described herein can be used for enrich regions of interested for determining the replication timing and the copy number variation (CNV) of genomic segments distributed throughout a genome of an organism. Comparing to whole-genome sequencing, Target enrichment using a capture library described herein considerably reduces time and expenses for analyzing replication timing and CNV. Certain illustrative embodiments of the invention are described below. The present invention is not limited to these embodiments.

Alteration in DNA copy number is one of the many ways in which gene expression and function may be modified. Some variations are found among normal individuals, some others occur in the course of normal processes in some species, and some others participate in causing various disease states. For example, many defects in human development are due to gains and losses of chromosomes and chromosomal segments that occur before or shortly after fertilization, and DNA dosage-alteration changes occurring in somatic cells are frequent contributors to cancer. Detecting these aberrations and interpreting them in the context of broader knowledge facilitates the identification of crucial genes and pathways involved in biological processes and disease. In addition, identification of polynucleotides that correspond to copy number alterations in cancerous, pre-cancerous, or low metastatic potential cells relative to normal cells of the same tissue type, provides the basis for diagnostic tools, facilitates drug discovery by providing for targets for candidate agents, and further serves to identify therapeutic targets for cancer therapies that are more tailored for the type of cancer to be treated.

Faithful transmission of genetic material to daughter cells involves a characteristic temporal order of DNA replication, which may play a significant role in the inheritance of epigenetic states. During the S-phase of each cell cycle, all DNA in a cell is duplicated in order to provide one copy to each of the daughter cells after the next cell division. The process of duplicating DNA is called DNA replication, and it takes place by first unwinding the duplex DNA molecule, starting at many locations called DNA replication origins, followed by an unzipping process that unwinds the DNA as it is being copied. However, replication does not start at all the different origins at once. Rather, there is a defined temporal order in which these origins start. Frequently a few adjacent origins open up to duplicate a segment of a chromosome, followed some time later by another group of origins opening up in an adjacent segment. Replication does not necessarily start at exactly the same origin sites every time, but the segments appear to replicate in the same temporal sequence regardless of exactly where within each segment replication starts.

The temporal order of replication of all the segments in the genome is called replication-timing program. At present, very little is known about either the mechanisms orchestrating the timing program or its biological significance. However, it is an intriguing cellular mechanism with links to many poorly understood features of the folding of chromosomes inside the cell nucleus. Replication timing is believed to be a critical epigenetic component of cellular function in addition to its role in the faithful replication of DNA sequence information. To ensure the transmission of cellular identity and function, the replication-associated inheritance of epigenetic templates must be faithfully copied as well as the sequence. The presence at the replication fork of chromatin assembly factors, histone-modifying enzymes, and DNA methyltransferases is consistent with this function. Additionally, the fidelity of DNA replication itself appears to be influenced by time of replication, because late domains exhibit increased mutation frequency.

All eukaryotes have a defined RT program that is largely conserved between closely related species, including humans and mice. Analyses of RT in various cell types have yielded insights into genome organization and repackaging events during development, suggesting an important role for the timing program itself or for 3D genome organization in regulating developmental gene expression. DNA replication in human cells proceeds according to a defined temporal order. In most cancers and in many diseases, the temporal order of replication is disrupted. Further research may reveal replication-timing changes as useful biomarkers for such diseases.

Overall, knowledge of CNV and Replication Timing provides an immediate clinical use in diagnosis and can, in some cases, provide useful prognostic information. Over the past several years, array Comparative Genomic Hybridization has proven its value for analyzing DNA copy-number variations and Replication Timing.

Comparative Genomic Hybridization (CGH) is a molecular cytogenetic method for analyzing CNVs relative to ploidy level in the DNA of a test sample compared to a reference sample, without the need for culturing cells. CGH is the first efficient approach to scanning the entire genome for variations in DNA copy number. In a typical CGH measurement, total genomic DNA is isolated from two sources to be compared, most commonly a test and a reference source such as a test cell populations and a reference cell populations; independently labeled with different fluorophores (fluorescent molecules) of different colors, such as red and green; denatured to single stranded DNA; mixed in a 1:1 ratio; and then hybridized to a normal metaphase spread of chromosomes, to which the labelled DNA samples will bind at their locus of origin. By application of a fluorescence microscope and computer software, the differentially colored fluorescent signals are then compared along the length of each chromosome for the identification of chromosomal differences between the two sources. A higher intensity of the test sample color in a specific region of a chromosome indicates the gain of material of that region in the corresponding source sample, while a higher intensity of the reference sample color indicates the loss of material in the test sample in that specific region. A neutral color (yellow when the fluorophore labels are red and green) indicates no difference between the two samples in that location. The relative hybridization intensity of the test and reference signals at a given location is then proportional to the relative copy number of those sequences in the test and reference genomes. If the reference genome is normal, increases and decreases in the intensity ratio directly indicate DNA copy-number variation in the genome of the test cells.

CGH is only able to detect unbalanced chromosomal abnormalities. This is because balanced chromosomal abnormalities, such as reciprocal translocations, inversions, or ring chromosomes, etc., do not affect copy number. CGH does, however, allow for the exploration of all 46 human chromosomes in a single test. The discovery of deletions and duplications, even on the microscopic scale, may be explored by other cytological techniques, leading to the identification of candidate genes.

Through the use of DNA microarrays in conjunction with CGH techniques, array comparative genomic hybridization (aCGH) has rapidly supplanted conventional metaphase CGH as the standard protocol for identifying segmental copy number alterations in disease state genomes. Many specific forms of aCGH have been developed, allowing for a locus-by-locus measure of CNV with an increased resolution as low as 100 kb. This improved technique allows for the etiology of known and unknown conditions to be discovered. To facilitate determinations of the relationship between therapeutic options and genomic aberrations, DNA microarrays designed to analyze targeted genomic regions relevant to chronic lymphocytic leukemia have been produced for using in clinical trials. Association of DNA copy-number aberrations with prognosis has been found for a variety of tumor types, including prostate cancer, breast cancer, gastric cancer and lymphoma.

Next generation sequencing platforms allow ultra-high-throughput massively parallel DNA sequencing at once and have sharply reduced the cost of sequencing. The next generation sequencing technologies therefore provide an alternative and adaptable method of detecting copy number, CNV-seq, by comparing the number of sequence reads in non-overlapping windows between patient and control samples. CNV-seq offers an alternative to array CGH for copy number analysis with a resolution and future costs comparable to conventional array CGH platforms and with less stringent sample requirements. Also, massively parallel DNA sequencing technologies also allow the ability to generate large amounts of sequencing data, including CNV and replication timing, at a rapid pace.

Repli Seq is a genome-scale approach to map temporally ordered replicating DNA using massively parallel sequencing. The replication-timing program generally can be measured by two different approaches. In one general approach, the amount of the different DNA sequences along the length of the chromosome per cell is literally measured. Sequences that duplicate first, long before the cell division, are more abundant in each cell than the sequences that replicate last just prior to the cell division. In the other approach, newly synthesized DNA is labeled by incorporating chemically tagged nucleotides into the newly synthesized DNA strands during synthesis, and then labeled newly synthesized DNA are purified from cells at different phase during the duplication process. In either case, the amount of the different DNA sequences along the length of the chromosome can be either measured directly using a machine that reads the amount of sequences which are present, or measured indirectly via microarray hybridization. In any case, the temporal order of replication along the length of each chromosome can be plotted in graphical forms to produce data of replication timing.

Traditionally, in sequencing a sample genome using microarray, copies of the sample genomic DNA are broken or sheared into short fragments. The short fragments are more or less randomly distributed across the sample genome. Each fragment of the short fragments is sequenced and the sequence of each fragment is then aligned to a reference sequence. All sequences of the short fragments are respectively aligned to reference sequences and are joined together to generate a complete genomic sequence of the sample genome.

To find the complete genomic sequence of a person by using a whole genomic microarray, it needs to sequence about 28 times (called 28×) of the person's genomic DNA. If only sequence the person's genomic DNA at an amount equivalent to the average of one times of the genome (1×), much of the sequence will be missed, because some genomic locations will be covered by several pieces that have been sequenced. Generally, the deeper the sequencing coverage, the more of the genome will be covered at least once. Since people are diploid; the deeper the sequencing coverage, the more likely that both chromosomes at a location will be included. Deeper coverage is particularly useful for detecting structural variants, and allows sequencing errors to be corrected.

For example, HiSeq2500 generates approximately 150M reads/lane for single-end rapid run mode. Assuming each read gives 50 nucleotides valid sequence, about 6.25-7.5 gigabase pairs (Gb), reads corresponding to approximately two times of (2×) human genome size are needed for a CNV analysis if a microarray is used. Assuming that sequencing reads distribute evenly, when windows having a size of 200 kb in length are used to smooth array data, there should be an average of approximately 1000 (=150×10⁶ reads×200×10³bp/3×10⁹ bp) reads in each 200 kb window from one lane.

In reality, about 50% of human genomic sequences are repetitive regions; thus, reads from those repetitive regions do not map. Assuming that each lane gives about 500 mapped reads for every 200 kb window, since each read has 50 bp in length, maximally only about 25 kb (500×50bp) out of 200 kb is actually covered by reads for lx depth sequence.

Microarray data on replicationdomain.org are scaled at a range of about +/−2.5 (log2), which corresponds to about 5.66 (=2²⁵). To guarantee the similar dynamic range and resolution (200 kb window with 10 kb sliding steps), approximately 100 total reads every 200 kb are needed for analyzing replication timing in E/L assay, and about 300 reads every 200 kb are needed for analyzing replication timing in S/G1 assay. Since only 0.5-1 sample can be loaded per lane, it is cost and time consuming to process many S/G1 samples that are required to diagnose disease.

However, genome-wide sequencing is still too expensive to deeply sequence many samples to find most genetic variants that have frequencies of at least 1% in the populations studied. In addition, in analyzing sequencing result, it is generally required to upload raw data as in FASTQ file to GEO for a manuscript to be accepted. Even if the cost for sequencing itself drops to 1/10, comparing to the cost of traditional sequencing, having high percentage of usable data, i.e., usable reads, in the original FASTQ file contributes to save the data storage space in the public database and save computation power for mapping (processing time with given number of CPU, necessary memory).

With targeted sequencing, only a subset of genes or defined regions in a genome is sequenced, allowing researchers to focus time, expenses, and data storage on the regions of interest. This approach may be used to sequence various individuals to discover, screen, or validate genetic variation within a population. Targeted NGS yields an increased depth of sequence over target regions and overall lowering cost per target. The ability to pool samples and obtain high sequence coverage during a single run allows NGS to identify rarer variants which are missed, or too expensive to identify, using conventional sequencing approaches.

In targeted sequencing, the fragments of interest are first capture enriched from a sequencing library by probes bound to certain capture materials, for example, magnetic beads. Once the unbound DNA in the library is washed away, fragments of interest captured by probes is eluted to provide a pool containing enriched fragments of interest.

Target-specific (user-defined) next generation sequencing, or targeted NGS, allows one to selectively capture genomic regions of interest from a DNA sample and to enrich sequences prior to the sequencing run. With the common goal to capture candidate genomic regions at high accuracy and completeness, while lowering the costs at the same time, several target enrichment strategies have been developed since the original description of the direct genomic selection (DGS) method in 2005. Widely applied strategies include methods such as multiplex PCR amplification, molecular inversion probes (MIP), hybrid capture, in-solution capture, etc. A common feature of these methods is their reliance on single-stranded oligonucleotides that serve as capture probes during target enrichment.

“Sequence capture” is a hybridization-based procedure to enrich the sequence/region of interest. By performing sequence capture to a sequence library (pool) before sequencing, only interested regions/genes from originally whole genome or transcriptome, etc., will be sequenced.

In hybrid capture, microarrays contain single-stranded oligonucleotides (oligos) with sequences from the human genome to tile the region of interest fixed to a surface. Genomic DNA is sheared to form double-stranded fragments. The fragments undergo end-repair to produce blunt ends and adaptors with universal priming sequences are added. These fragments are hybridized to oligos on the microarray. Unhybridized fragments are washed away and the desired fragments are eluted. The fragments are then amplified using PCR. Limitations of the hybrid capture include the need for expensive hardware as well as a relatively large amount of DNA.

Solution-based capture is a target enrichment strategy to capture genomic regions of interest in which a pool of oligonucleotides, i.e., capture probes or capture oligos, is synthesized and hybridized in solution to a fragmented genomic DNA sample. Capture oligos may selectively hybridize to DNA fragments of interest. After hybridization, capture substrates such as beads may be added into the mixture of hybridization to bind on capture oligos. DNA fragments of interest hybridized to capture oligos bound on the beads can be pulled down and be washed to clear excess material. The DNA fragments of interest captured by the capture oligos can then be subject to high-throughput sequencing, allowing for selective sequencing of genomic regions of interest. Solution-based capture, as opposed to hybrid capture, applies excess of probes to target regions of interest over the amount of template required. Selecting a preferred method is dependent on several factors including: number of base pairs in the region of interest, demands for reads on target, equipment in house, etc.

Various approaches to capture sequences of interest before sequencing are reported for processing S/G1 or E/L repli-seq data. For example, according to one reported approach, segments covered by 200 reads in the G1 fractions are defined as windows and S phase reads in the same windows are counted.² The average size of these segments are about 2 kb. Data are normalized to 0 mean and 1 SD. Then 100 kb windows of 100 kb along the chromosomes that contain data segments with an internal standard deviation larger than 1.1 are removed from further analysis, which correspond predominantly to borders of centromeric gaps. The reported methods starts from x0.9-8.9 depth/fraction (G1 or S) and the average read number out of 1 kb is about 100. As a result, if per fraction needs to load 2 lanes, approximately 3×10⁸ reads are needed for each fraction. ²

In another reported approach, 5 kb windows are created for each of the S and G1 files and the hit numbers in them are counted. A filter is then applied to eliminate windows with less than 10 hits in the G1 library and the S/G1 ratio is calculated.³ The number of hits is then smoothed and the data is processed to obtain repli-seq data. According to the report, more than 10 million reads are uniquely mapped to the genome. Chromosome-wide TimEX-seq profiles are obtained based on 10.5 million (basophilic erythroblasts) and 13 million reads (hESC). To determine the minimal number of reads necessary to obtain timing profiles, the above calculations using decreasing amounts of reads are repeated. Timing profiles that are well correlated with the profiles obtained with 10 million reads (r=0.95) could be obtained with as little as 5 million reads. Datasets containing less than 1.5 million tags do not yield reliable TimEX data.³

Comparing to whole-genome sequencing, targeted NGS focuses on clinically actionable genes at both higher quality and lower cost. The main challenge for clinical targeted sequencing is to obtain a complete and uniform coverage of all target regions. The technology in the embodiments of the present invention couples probe design with solution-based capture-sequencing to develop a product and method that aim at obtaining a complete and uniform coverage of all target regions and reducing sequencing cost. The product and method disclosed herein will replace traditional Comparative Genomic Hybridization (CGH) for characterization and diagnosis of patient samples for Copy Number Variation (CNV) and Replication Timing, as well as for basic research applications.

### Design of Capture Library

One concern in selecting an aCGH platform for gene discovery is usually the minimal size of the genomic region having altered copy number that can be reliably detected. This concern also exists in design of capture probes for sequence capture. In design of capture library, both upper and lower resolution limitations should be calculated. Resolution means capture spacing. Usually, the upper resolution limitation is where the cost of creating a capture library meets the savings in sequencing. The lower resolution limitation is the need of probe spacing for different applications. Different capture spacing may be defined according to different specific applications such as CGH, pre-natal diagnosis, etc. For example, the minimal size of genomic region having altered copy number may be about 6-12 kb, which is generally provided by microarray platform and leads to satisfactory replication timing data. In one example of the present invention, the minimal size of genomic region having altered copy number may be about 10 kb.

Accordingly, some embodiments of the present invention develop a solution-based sequence capture method enabling the enrichment of regions of interest every 10 kilobases (kb) of a genome and ensuring the sequencing of genomic information at evenly-spaced locations across the genome. The regions of interest may comprise about 150 base pairs (bp) in length, resulting in a resolution that is close to the spacing between the enriched regions on the genome.

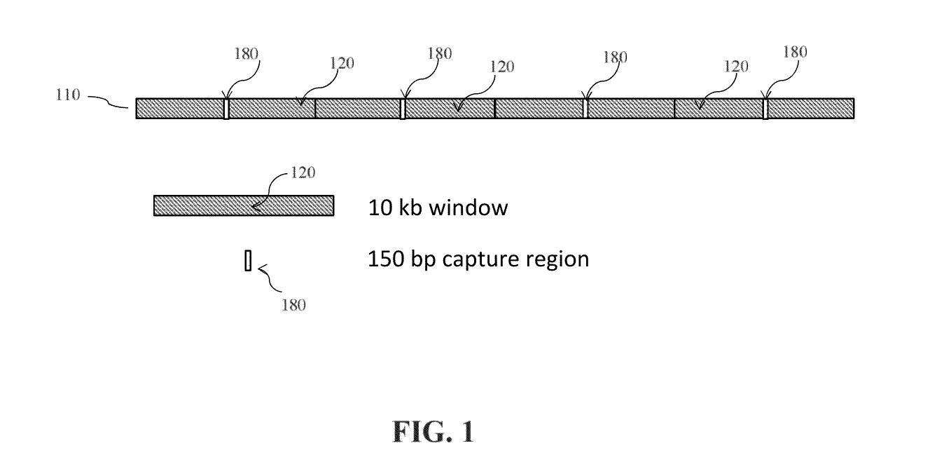

FIG. 1 is an example of a plurality of capture regions evenly-spaced along a genome. A genome region 110 under study is evenly divided into consecutive windows 120, wherein each of the consecutive windows 120 comprises about 10 kb in length. Each of consecutive windows contains a capture region 180. Each capture region 180 has a size of about 150 bp in length. As a result, a plurality of capture regions 180 is approximately evenly-spaced along genome 110, wherein each capture region 180 resides within one of consecutive windows 120 along genome 110. Each two adjacent capture regions of the plurality capture regions are separated by a spacing of about 10 kb in length.

According to some embodiments, the size of a plurality of capture regions may not be limited to 150 bp in length. A plurality of capture regions may have about 68 bp to about 196 bp in length. Further, the spacing, i.e., distance, between two adjacent capture regions may be various. For example, the spacing between two adjacent capture regions may be about 6 to about 14 kilobases.

To further define the spacing between two adjacent capture regions and the location of capture regions on a genome, a plurality of target regions along a genome are introduced. The size of the plurality of target regions may be determined according to the need for resolution. In some embodiments of the present invention, the size of the plurality of target regions is about 4 kb in length. Each of the plurality of targets regions may be chosen automatically in the middle of one window of the plurality of consecutive windows along a genome. Therefore, each target region of the plurality of target regions may be located approximately at a central region of one window of the plurality of consecutive windows along a genome. As a result, the plurality of target regions is approximately evenly-spaced along the genome. Further, each of the plurality of target regions may contain one or more non-repetitive regions.

According to embodiments, each capture region of a plurality of capture regions resides within one target region of the plurality of target regions. A capture region of the plurality of capture regions may reside at any location within a target region of the plurality of target regions. For example, a capture region of the plurality of capture regions may reside at a central region of one target region of the plurality of target regions, or may reside near an end region of a target region of the plurality of target regions. A capture region of the plurality of capture regions is chosen from a non-repetitive region within one of the plurality of target regions. When multiple non-repetitive regions exist within one of the plurality of the target regions, the non-repetitive region near the center of the one of the plurality of the target regions is chosen as a capture region. As a result, the spacing between two adjacent capture regions of the plurality of capture regions may be about 6 kb to about 14 kb in length. In some embodiments, the average spacing between two adjacent capture regions of the plurality of capture regions is about 10 kb in length.

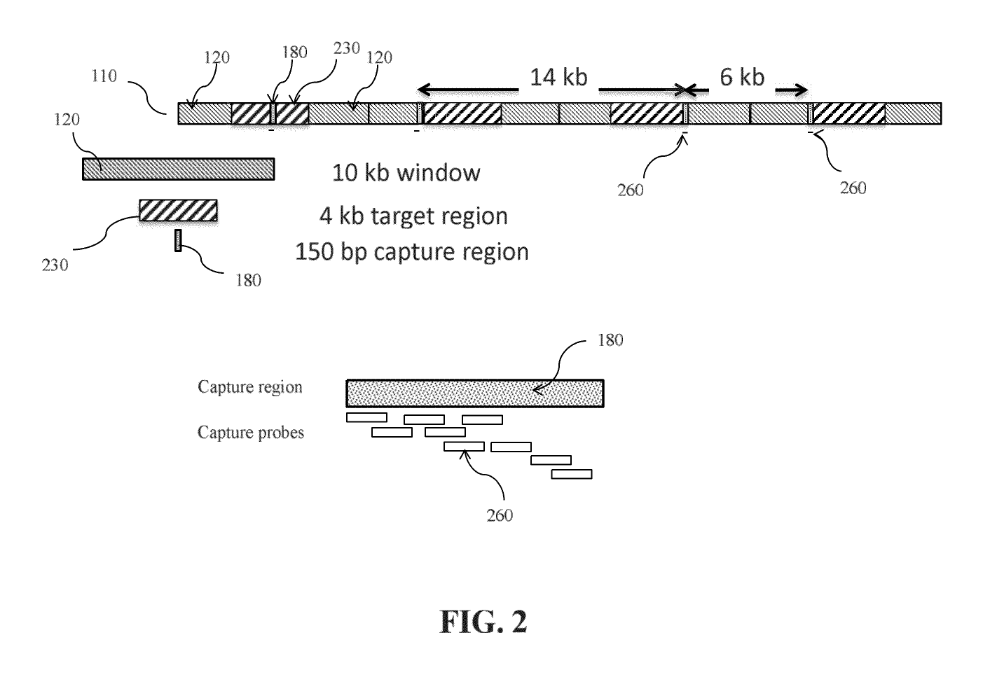

FIG. 2 shows an example of a plurality of target regions. In the example, each window of a plurality of consecutive windows 120 has a size of 10 kb in length along genome 110. Each window of the plurality of consecutive windows 120 encompasses a target region 230. Each target region 230 is 4 kb in length and is located at the central region of one window of the plurality of consecutive windows 120 along genome 110. As a result, the plurality of target regions 230 is approximately evenly-spaced along genome 110. In addition, the plurality of target regions 230 may comprise one or more non-repetitive regions.

The size of a plurality of target regions shown in FIG. 2 is only an example in some embodiments. The size of a plurality of target regions is not limited to 4 kb in length.

Further, as shown in FIG. 2, each target region 230 contains one capture region 180. A capture region 180 may reside at a central region or near an end region or a target region 230, or at any other location within a target region 230. The spacing between two adjacent capture regions 180 may be about 6 kb to about 14 kb in length. The average spacing between two adjacent capture regions 180 may be about 10 kb in length. As a result, the plurality of capture regions 180 is approximately evenly-spaced along genome 110, with an average spacing of about 10 kb in length.

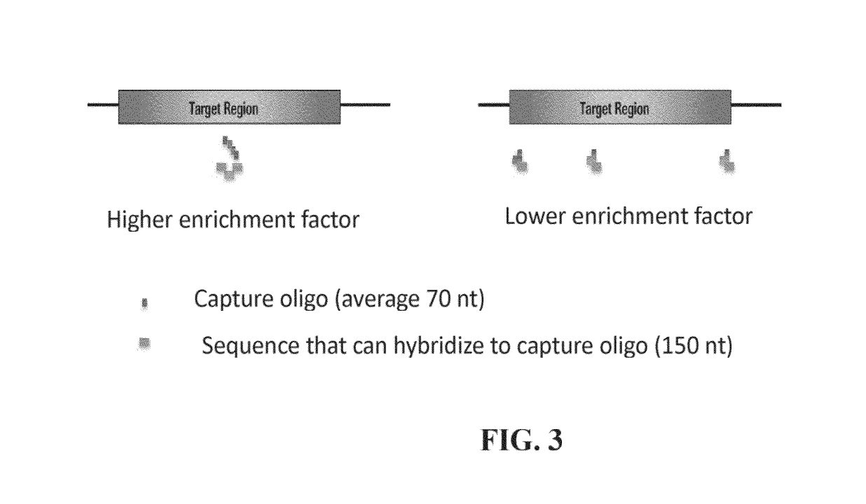

FIG. 2 further shows a plurality of capture oligos 260, or capture probes, designed to be able to hybridize their complementary DNA sequences within the plurality of capture regions 180 in genome DNA 110, so the plurality of capture oligos 260 is enable to capture the plurality of capture regions 180 from a genomic DNA. To achieve a high enrichment outcome, the plurality of capture oligos is preferred to overlap each other and tile the plurality of capture regions. Comparing to a set of plurality of capture oligos scattered in a target region as shown in FIG. 3, a set of capture oligos that tiles a portion of a target region results in a higher enrichment factor and fewer off-target reads. Accordingly, in embodiments of the present invention, a plurality of capture oligos in a capture library is designed to tile a plurality of capture regions along a genome. More specifically, a plurality of sets of capture oligos is encompassed within a capture library. Each set of capture oligos tiles one capture region of a plurality of capture regions. Each capture oligo of the plurality of capture oligos is able to bind to a fragment of sequence within a capture region of the plurality of caption regions. For example, as shown in FIG. 2, in some embodiment, a set of capture oligos 260 tiles one capture region 180. According to some embodiment of the present, the size of a plurality of capture oligos may be about 50 nucleotides to about 105 nucleotides (nt). In some embodiments, a desired size of a plurality of capture oligos is about 70 nt in length.

Different capture library comprising a plurality of capture oligos may target genome DNA of different source or species. For example, a plurality of capture oligos may target DNA sequences from a human genome or from a non-human genome. As a result, a plurality of capture oligos tiling a plurality of capture regions approximately evenly-spaced along a human genome is able to capture regions of interest from genomic DNA fractions of a sample from a person. A plurality of capture oligos tiling a plurality of capture regions approximately evenly-spaced along a non-human genome is able to capture regions of interest from genomic DNA fractions of a non-human sample.

According to embodiments of the present invention, a capture library may be custom-ordered, with above-described requirements.

Further, a product encompassing a capture library described herein may be a kit that contains a plurality of capture oligos and capture substrates such as beads. The capture substrates may be streptavidin-coated beads or streptavidin-coated magnetic beads. Each of the plurality of capture oligos may be labeled with biotin (biotinylated). In some embodiments, a plurality of biotinylated capture oligos may be coupled to streptavidin-coated beads or streptavidin-coated magnetic beads and be used as capture probes to hybridize target regions in target enrichment. In some embodiments, a plurality of biotinylated capture oligos is separated with streptavidin-coated beads or streptavidin-coated magnetic beads in a kit.

### Target Enrichment

The capture library described herein can be a comparative genomic hybridization capture library for targeted sequencing. According to some embodiments, the capture library described herein may be applied in a solution-based capture to enrich regions of interest from genomic DNA. Accordingly, embodiments of the present invention provide methods of using the capture library described herein to obtain one or more pools of DNA fragments of interest. The methods in some embodiment comprise performing target enrichment for one or more sample sequencing libraries comprising total genomic DNA fractions with the use of the capture library described herein.

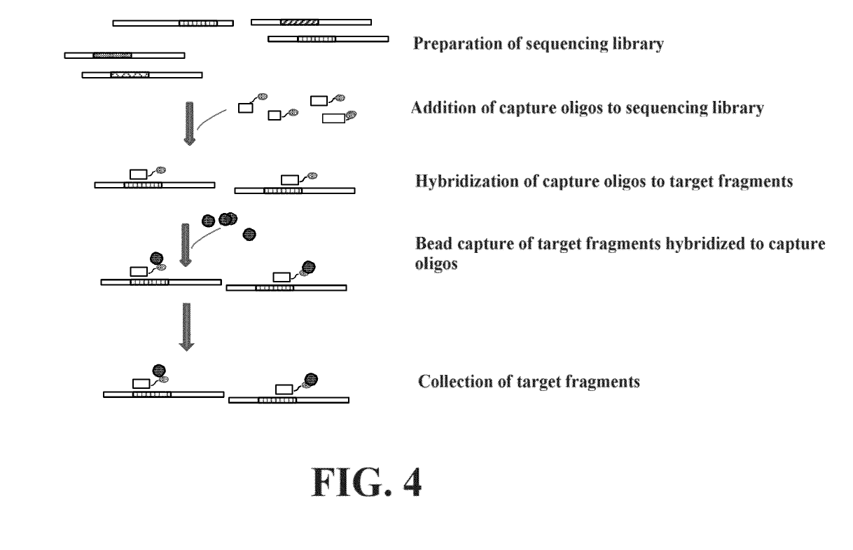

FIG. 4 illustrates an exemplary enrichment process using a capture library described herein. In some embodiments, a sequencing library of a sample is prepared by shearing genomic DNA of the sample into small fragments to generate genomic DNA fractions of the sample. The small fragments are denatured into single strands for hybridizing in a hybridization solution to a capture library which comprises a plurality of biotinylated capture oligos described herein to capture fragments having sequences that are complementary to the sequences in capture oligos. After hybridization, streptavidin-coated magnetic beads are added into the hybridization solution to bind to biotinylated capture oligos. Further, the small fragments that are not hybridized to capture oligos are washed away and the small fragments that are captured by the capture oligos are collected to form a pool of fragments comprising sequences of interest.

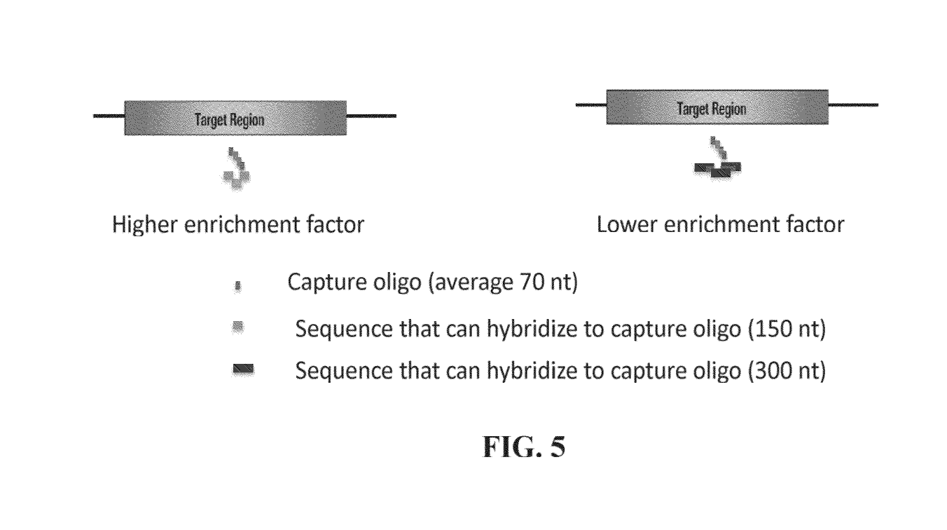

As an example shown in FIG. 5, in some embodiments, to make sequencing library comprising genomic DNA fractions, genomic DNA of a sample may be sheared into small fragments comprising 1 bp—to about 300 bp. In some embodiment, the small fragments have an average size of about 150 bp in length. Then, the small fragments are ligated with certain common DNA sequences on both ends of shared DNA fragments. As a result, the size of the ligated DNA fragments in the library is about 150-450 bp (average 300 bp). Then the library is denatured to single strand and hybridized to capture oligos for target enrichment.

Compared with small fragments having a size of 300 bp (before be ligated with certain common DNA sequences), small fragments having a size of 150 bp (before be ligated with certain common DNA sequences) provide higher enrichment factor. For example, as shown in FIG. 5, capture oligos with an average size of 70 nucleotides (nt) in length are used to capture target fragments. The capture oligos can hybridize to a capture region contained within a 150 nt fragment and a 300 nt fragment (single-strand DNA after denatured for hybridization), respectively. Comparing to the 300 nt fragment, the 150 nt fragment provides less unwanted region after target enrichment using the capture oligos and therefore leads to higher enrichment factor. Therefore, in a following describe example, sequencing libraries comprise small fragments of about 150 bp in length.

The success of the enrichment of genomic DNA fragment pool may be measured by qPCR at control loci. The enriched pool of the small fragments of interest can be then subject to high-throughput sequencing for analyzing replication timing and copy number variation (CNV). In some embodiments, the capture library described herein is used for analyzing replication timing to human pediatric acute lymphocytic leukemia samples. The capture library described herein reduces the sequence space by approximately 99% and ensures the sequencing of genomic information at evenly-spaced locations across the genome, providing a resolution close to the spacing between two adjacent capture regions, for example, about 10 kb in some embodiments.

According to embodiments, a method for analyzing replication timing or CNV includes performing target enrichment for one or more sequencing libraries using a capture library described herein to obtain one or more pools of enriched DNA fragments of interest. Enriched DNA fragments of interest are subsequently subject to high-throughput sequencing for analyzing replication timing or CNV.

In some embodiments, a sequencing library of “early S phase” (E) and a sequencing library of “late S phase” (L) are prepared from cells at “early S phase” and from cells at “late S phase,” respectively. In some embodiments, small genomic DNA fragments comprised within the sequencing library of “early S phase” and within the sequencing library of “late S phase” are sheared into small fragments to form genomic DNA fractions, and the small fragments comprise up to about 300 bp in length, wherein the small fragments may have an average length of about 150 bp. The sequencing libraries of “early S phase” and “late S phase” are further enriched via the application of a capture library described herein to generate separate pools of enriched DNA fragments for “early S phase” and “late S phase” samples. The enriched DNA fragments for “early S phase” and “late S phase” samples comprise sequences that are complementary to the plurality of capture probes in the capture library. The Enriched DNA fragments for “early S phase” and “late S phase” samples are subsequently subject to high-throughput sequencing for analyzing “early S/late S” (E/L) replication timing.

In some embodiments, a sequencing library of “S phase” and a sequencing library of “G1 phase” are prepared from cells at “S phase” and from cells at “G1 phase,” respectively. In some embodiments, small genomic DNA fragments comprised within the sequencing library of “S phase” and within the sequencing library of “G1 phase” may be up to 300 bp in length. In some embodiments, small genomic DNA fragments comprised within the sequencing library of “S phase” and within the sequencing library of “G1 phase” are approximately 150 bp in length. The sequencing libraries of “S phase” and “G1 phase” are further enriched via the application of a capture library described herein to generate separate pools of enriched DNA fragments for “S phase” and “G1 phase” samples. Enriched DNA fragments for “S phase” and “G1 phase” samples comprise sequences complementary to the plurality of capture probes in the capture library. The enriched DNA fragments for “S phase” and “G1 phase” samples are subsequently subject to high-throughput sequencing for analyzing of interest are subsequently subject to high-throughput sequencing for analyzing “S/G1” replication timing.

The present invention is further defined in the following Examples. It should be understood that these Examples are given by way of illustration only. From the above discussion and these Examples, one skilled in the art can ascertain the essential characteristics of embodiments of the present invention. Without departing from the spirit and scope thereof, one skilled in the art can make various changes and modifications of the invention to adapt it to various usages and conditions. All publications, including patents and non-patent literature, referred to in this specification are expressly incorporated by reference herein.

### EXAMPLE

### Example 1

### Create Custom-Order Capture Library

This example is an illustration of creating capture library via custom-design order. This example compares commercialized genome-wide CGH arrays with the capture library designed according to the embodiments of the current invention.

Many CNV arrays having a high density of probes in the target regions are commercially available. However, in many of these formats, probes are scattered genome-wide but NOT evenly-spaced. Differently, capture probes described in the present invention are evenly-spaced along a genome. To obtain a capture library comprising capture probes tiling capture regions evenly-spaced along genome for measuring replication timing, consecutive windows with a size of about 10 kb are selected. Sequences representing about 150 bp regions from about every 10 kb are further selected as capture regions. By such design, the capture library allows sequencing only 1/40 of the genome to obtain the same dynamic range of replication timing.

Custom-design capture library may be ordered from a database such as Roche NimbleGen, Agilent Technologies, etc. In this example, the orders are made to Roche NimbleGen database. Roche NimbleGen provides several platforms, for example, SeqCap EZ Choice Library, SeqCap EZ Choice XL Library, SeqCap EZ Developer Library, etc. SeqCap EZ Choice XL Library provides about 7 mb-200 Mb human genomic regions and can be used for capturing from more than about 7 Mb up to about 50 Mb of genomic regions. SeqCap EZ Developer Library provides up to about 200 Mb regions from human or non-human genomic DNA.

The capture library obtained by the above described order is useful in targeted sequencing newly replicated DNA (Repli-seq) and in analyzing CNV/CGH. At least an 8 fold reduction in cost to carry out CGH analysis using next generation sequencing (NGS) may be achieved by this capture library described in this example.

### Example 2

### Application of the Capture Library in Replication Timing Assay

The example illustrates the application of a capture library disclosed herein to enrich regions of interest for running next generation sequencing for obtaining replication timing information. In an exemplary embodiment, this technology may be similar to “Comparative Genomic Hybridization (CGH)” whereby a test genome and a reference genome are hybridized to capture probes tiling evenly-spaced capture regions along a genome.

In this example, a pool of enriched fragments containing interested capture regions are generated before running sequencing for replication timing. By using the capture library disclosed herein, a solution-based sequence capture enables the capture of an equal amount of sequences with a spacing of about every 6 kb to about 14 kb along the genome to achieve an even coverage of the genome without using microarray. For example, representative 150 bp regions from every 10 kb of a genome DNA are captured during enrichment, resulting only 1/40 of the genome being sequenced for obtaining the same dynamic range of replication timing as being obtained by using a microarray. The technology of the present invention provides an approach to obtain satisfactory dynamic range without relying on massive sequencing and sophisticated computation.

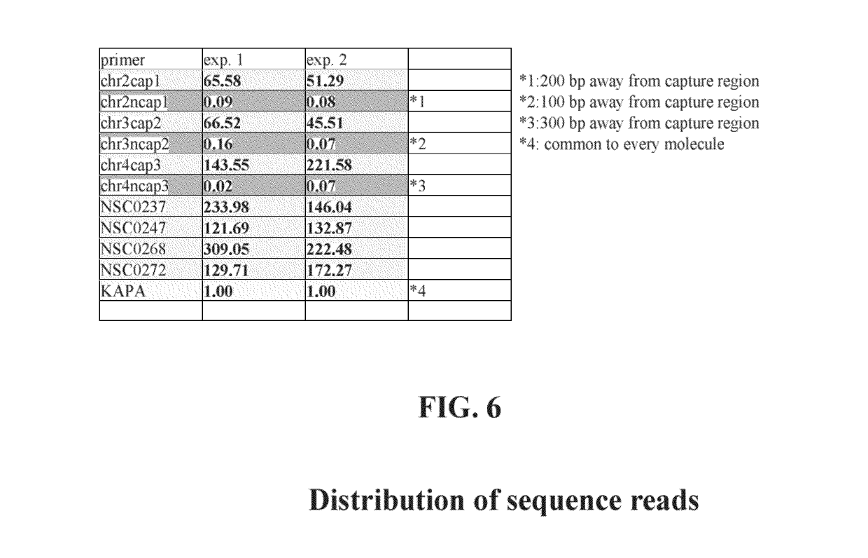

FIG. 6 is a graph of relative quantity for target and non-target loci of pre-/post-capture samples (showing post-capture enrichment evaluation by qPCR) according to one embodiment of the present invention. Exp. 1 and Exp. 2 are from different library pools, and the results are not exactly the technical repeats using the same materials. The “fold enrichment” for each locus is calculated by deltaCt (difference of Ct value between pre- and post-capture samples) and E value (amplification efficiency for each primer set in the used PCR condition) of qPCR, then normalized against primer set “KAPA”.

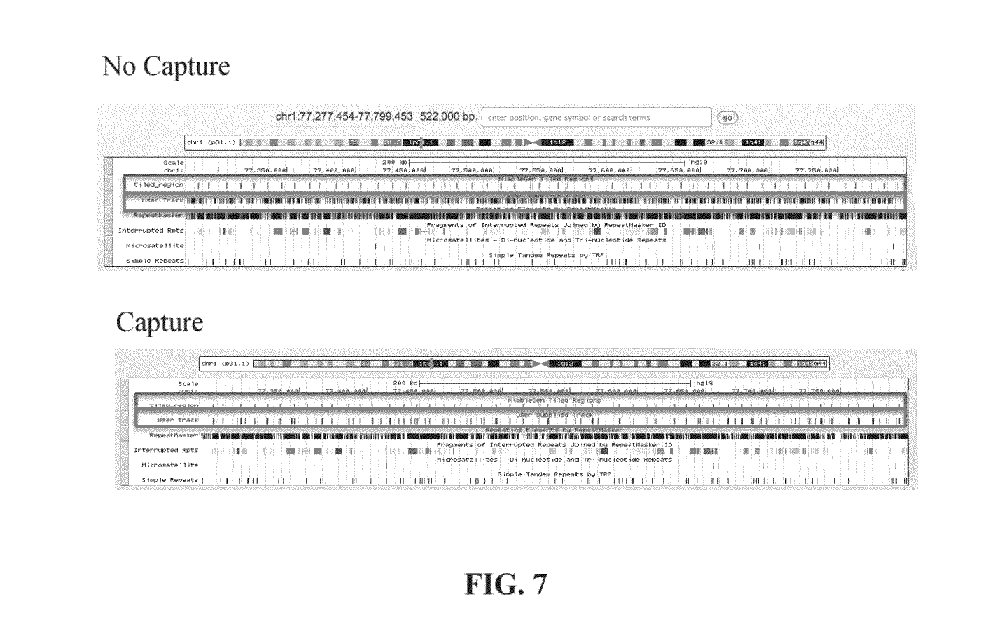

FIG. 7 shows screenshots from UCSC Genome browser with custom tracks (capture regions and actual reads) (showing distribution of sequence reads with and without capture; reads from non-captured sample distribute randomly while reads from captured sample distribute in 150 bp capture region shown in FIG. 1 according to one embodiment of the present invention. These figures are obtained by uploading custom tracks of capture library design and sequence reads, to UCSC genome browser (http://genome.ucsc.edu/). The horizontal axis shows genomic loci. The vertical axis in this mode is just plus (exists) or minus (non-exists) of each feature at shown loci, hence “Y-axis” is not labeled from its nature. In FIG. 7, vertical bars in upper box in each screenshot show regions to be captured and vertical bars in lower box in each screenshot show actual reads obtained.

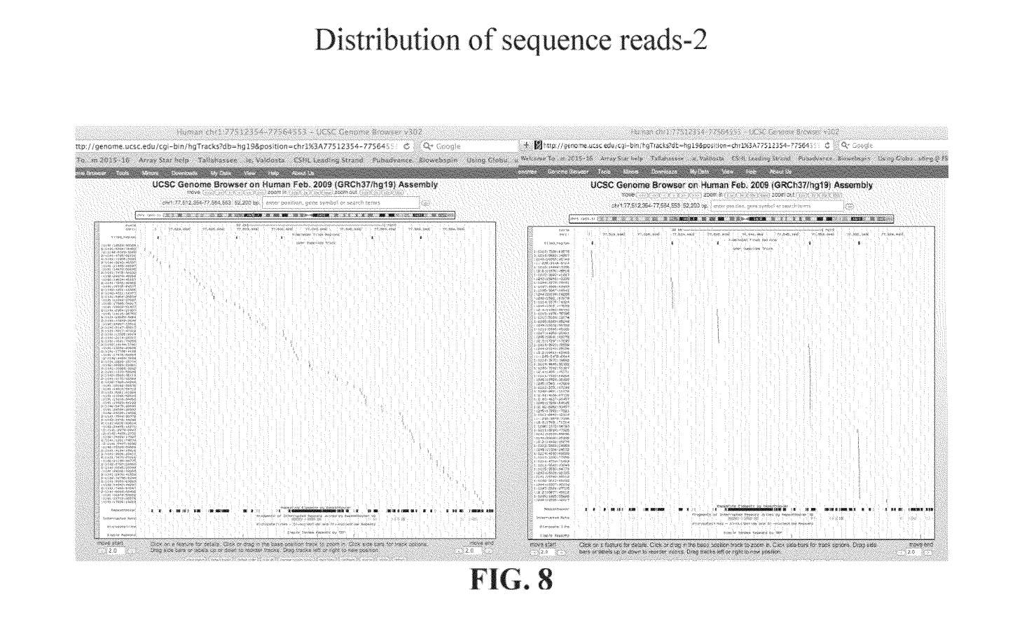

FIG. 8 shows an enlarged representative region from FIG. 7 (distribution of sequence reads) according to one embodiment of the present invention.

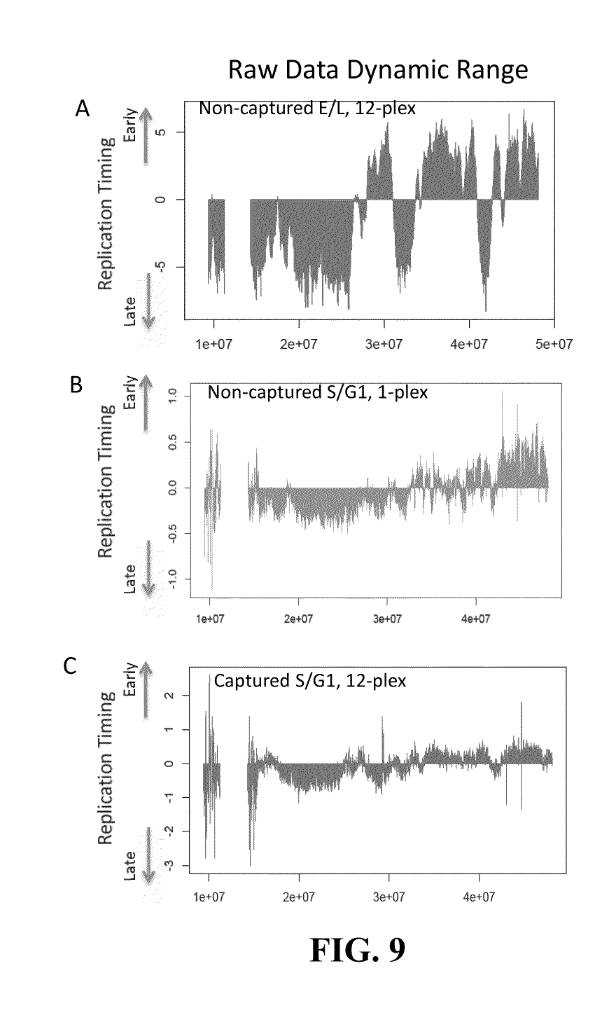

FIG. 9 shows a comparison of raw data (before scaling) dynamic range from E/L (FIG. 9, Panel A), non-capture S/G1(FIG. 9, Panel B), captured S/G1 repli-seq (FIG. 9, Panel C). Raw data dynamic range is one of the greatest factors that contribute to the signal-to-noise ratio of this assay. The E/L method intrinsically has a greater dynamic range than the S/G1 method. Disclosed embodiments of the invention facilitate an increase to the dynamic range of the S/G1 method in a cost-efficient manner. “Replication Timing” is calculated according to the following formulas:

$$\begin{matrix}\frac{S}{G 1} replication timing = log 2 \frac{Reads / million \text{/} 50 kb from S}{Reads / million \text{/} 50 kb from G 1 )} & ( 1 ) \\ \frac{E}{L} replication timing = log 2 \frac{Reads / million \text{/} 50 kb from E}{Reads / million \text{/} 50 kb from L} & ( 2 )\end{matrix}$$

Methods and techniques, including techniques and methods conducted physically and/or by a computer, and may be employed in various embodiments of the present invention to analyze replication timing and CNV of a sample and for diagnosis of specific type of leukemic cells based on copy number variation or replication timing information.

### Example 3

### A Kit Containing a Capture Library

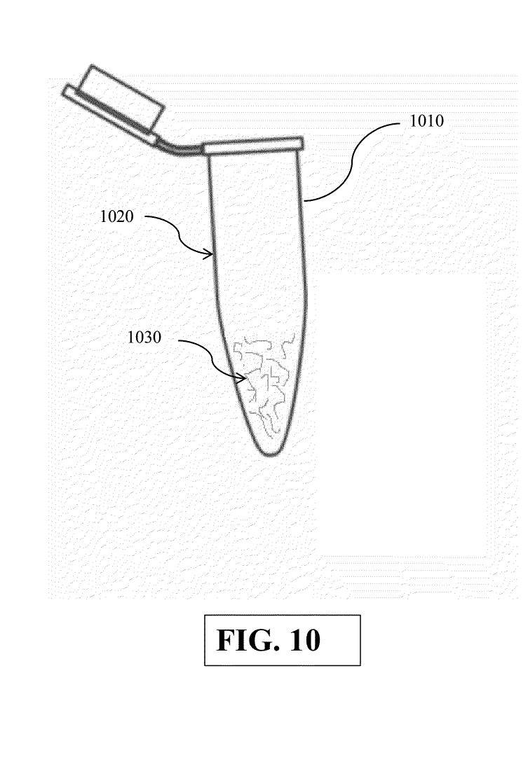

FIG. 10 shows an example of a kit comprising a capture library contained within a container according to some embodiments of the present invention. A kit 1010 comprises a container 1020 and a capture library comprising a plurality of capture oligos 1030. In one example, capture oligo 1030 contained in the container 1020 may be biotinylated. Biotinylated capture oligos may be bound to capture material such as streptavidin-coated magnetic beads for pulling target sequences hybridized to the biotinylated capture oligos.

In some embodiments, a plurality of biotinylated capture oligos is separately packaged with streptavidin-coated beads or streptavidin-coated magnetic beads in a kit.

The above kit is only an example of different kits. The container for the kits is also not limited to the container illustrated herein. Any appropriated containers that can store oligos are suitable. The plurality of capture oligos in a kit may be in the form as dry powder, wherein the plurality of capture oligos may be dissolved just before being used in target enrichment. In some example, the plurality of capture oligos is in a solution, wherein the plurality of capture oligos are dissolved in appropriated solution such as appropriate buffer solution.

All documents, patents, journal articles and other materials cited in the present application are incorporated herein by reference.

Having described a particular embodiment of the present invention, it will be apparent that modifications and variations are possible without departing from the scope of the invention defined in the appended claims. Furthermore, it should be appreciated that the example provided in the present disclosure, while illustrating a particular embodiment of the invention, is provided as a non-limiting example and is, therefore, not to be taken as limiting the various aspects so illustrated. It is intended that the invention not be limited to the particular embodiment disclosed herein contemplated for carrying out this invention, but that the invention will include all embodiments falling within the scope of the claims.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

All documents, patents, journal articles and other materials cited in the present application are incorporated herein by reference.

### REFERENCES

The following references are referred to above and are incorporated herein by reference:
1. Koren A, McCarroll S A. Random replication of the inactive X chromosome. Genome Res. 2014 Jan; 24(1):64-9. doi: 10.1101/gr.161828.113. Epub 2013 Sep 24.PMID: 24065775.
2. Koren A, Polak P, Nemesh J, Michaelson J J, Sebat J, Sunyaev S R, McCarroll S A. Differential relationship of DNA replication timing to different forms of human mutation and variation. Am J Hum Genet. 2012 Dec 7; 91(6):1033-40. doi: 10.1016/j.ajhg.2012.10.018. Epub 2012 Nov 21.PMID: 23176822.
3. Desprat R, Thierry-Mieg D, Lailler N, Lajugie J, Schildkraut C, Thierry-Mieg J, Bouhassira E E. Predictable dynamic program of timing of DNA replication in human cells. Genome Res. 2009 Dec;19(12):2288-99. doi: 10.1101/gr.094060.109. Epub 2009 Sep 18. PMID:19767418.
4. Audit B, Baker A, Chen C L, Rappailles A, Guilbaud G, Julienne H, Goldar A, d'Aubenton-Carafa Y, Hyrien O, Thermes C, Arneodo A. Multiscale analysis of genome-wide replication timing profiles using a wavelet-based signal-processing algorithm. Nat Protoc. 2013 Jan; 8(1):98-110. doi: 10.1038/nprot.2012.145. Epub 2012 Dec 13 .PMID:23237832.
5. Baker A, Audit B, Chen C L, Moindrot B, Leleu A, Guilbaud G, Rappailles A, Vaillant C, Goldar A, Mongelard F, d'Aubenton-Carafa Y, Hyrien O, Thermes C, Arneodo A. Replication fork polarity gradients revealed by megabase-sized U-shaped replication timing domains in human cell lines. PLoS Comput Biol. 2012;8(4):e1002443. doi: 10.1371/journal.pcbi.1002443. Epub 2012 Apr 5. PMID:22496629.
6. Guilbaud G, Rappailles A, Baker A, Chen C L, Arneodo A, Goldar A, d'Aubenton-Carafa Y, Thermes C, Audit B, Hyrien O. Evidence for sequential and increasing activation of replication origins along replication timing gradients in the human genome. PLoS Comput Biol. 2011 Dec;7(12):e1002322. doi: 10.1371/journal.pcbi.1002322. Epub 2011 Dec 29. PMID:22219720.
7. Chun-Long Chen, Aurélien Rappailles, Lauranne Duquenne, Maxime Huvet, Guillaume Guilbaud, Laurent Farinelli, Benjamin Audit, Yves d'Aubenton-Carafa, Alain Arneodo, Olivier Hyrien, and Claude Thermes. Impact of replication timing on non-CpG and CpG substitution rates in mammalian genomes. Genome Res. 2010 April; 20(4): 447-457. doi: 10.1101/gr.098947.109 PMCID: PMC2847748.
8. Hansen R S, Thomas S, Sandstrom R, Canfield T K, Thurman R E, Weaver M, Dorschner M O, Gartler S M, Stamatoyannopoulos J A. Sequencing newly replicated DNA reveals widespread plasticity in human replication timing. Proc Natl Acad Sci USA. 2010 Jan 5; 107(1):139-44. doi: 10.1073/pnas.0912402107. Epub 2009 Dec 4.PMID:19966280.
9. Moindrot B, Audit B, Klous P, Baker A, Thermes C, de Laat W, Bouvet P, Mongelard F, Arneodo A. 3D chromatin conformation correlates with replication timing and is conserved in resting cells. Nucleic Acids Res. 2012 Oct; 40(19):9470-81. doi: 10.1093/nar/gks736. Epub 2012 Aug 8. PMID:22879376.

What is claimed is:

1. A product comprising:
a capture library comprising a plurality of capture oligos,
wherein the plurality of capture oligos tiles a plurality of capture regions approximately evenly-spaced along a genome,
wherein the plurality of capture regions comprises about 68 to about 196 base pairs (bp) with an average of about 150 bp,
wherein each two adjacent capture regions of the plurality capture regions are separated by a spacing of about 6 to about 14 kilobases (kb), and
wherein the product is a comparative genomic hybridization capture library.

2. The product of claim 1, wherein each capture region of the plurality of capture regions resides within one target region of a plurality of target regions, wherein each target region of the plurality of target regions resides approximately at a central region of one window of a plurality of consecutive windows along the genome, wherein each of the plurality of consecutive windows has a size of about 10 kb.

3. The product of claim 2, wherein each of the plurality of capture regions comprises a non-repetitive region within one window of the consecutive windows along the genome.

4. The product of claim 2, wherein each target region of the plurality of target regions comprise about 4 kb in length.

5. The product of claim 4, wherein each capture region of the plurality of capture regions resides anywhere within a target region of the plurality of target regions.

6. The product of claim 1, wherein each capture oligo of the plurality of capture oligos comprises about 50 to about 105 nucleotides in length.

7. The product of claim 6, wherein the plurality of capture oligos comprises an average length of about 70 nucleotides (nt).

8. The product of claim 1, wherein the genome is a human genome.

9. A method comprising: performing target enrichment to one or more sequencing libraries using the product of claim 1 to obtain one or more pools of DNA fragments of interest captured by the plurality of capture oligos of the capture library, wherein each sequencing library of the one or more sequencing libraries comprises total genomic DNA fractions of a sample.

10. The method of claim 9, wherein the method further comprises shearing genomic DNA into small fragments, and wherein the small fragments comprise 1 bp—about 300 bp.

11. The method of claim 10, wherein the small fragments have an average length of about 150 bp.

12. The method of claim 9, wherein the pools of DNA fragments of interest captured by the plurality of capture oligos during target enrichment are subjected to high-throughput sequencing for analyzing copy number variation or replication timing.

13. The method of claim 9, wherein the genomic DNA fractions are prepared from cells at “S phase” and from cells at “G1 phase,” and wherein the one or more pools of DNA fragments of interest are subject to high-throughput sequencing for analyzing “S/G1” replication timing.
